(19) United States
(12) Patent Application Publication
(10) Pub. No.: US 2007/0154364 A1
(43) Pub. Date: Jul. 5, 2007

(54) Sterilizing Instrument

(75) Inventors: Kuang-Tai Tseng, Yonghe City (TW); Kuo-Kang Chen, Taipei (TW)

Correspondence Address: KAMRATH & ASSOCIATES P.A., 4825 OLSON MEMORIAL HIGHWAY, SUITE 245, GOLDEN VALLEY, MN, 55422, US

(73) Assignee: Rosace International Co., Ltd., Taipei (TW)

(21) Appl. No.: 11/324,402

(22) Filed: Jan. 3, 2006

(51) Int. Cl. A61L 2/18; B01J 19/08

(52) U.S. Cl. 422/186.07; 422/292

(57) ABSTRACT

A sterilizing instrument has a pump to boost ozone solution pressure, so that a large amount of ozone is dissolved in the water. The ozone solution is further processed by a depressurizer to generate bubbles in nanometer scale, which contains oxyhydrogen free radicals that can destroy bacteria and virus and is free to pollution to the environment.

### BACKGROUND OF THE INVENTION

1. Field of the Invention

The present invention relates to a sterilizing instrument, and particularly relates to a sterilizing instrument to provide a sterilizing effect depending on free radicals generated by dissolved ozone.

2. Description of the Related Art

It is always necessary to clean and sterilize food, food receptacles, and medical apparatuses. In a conventional way, objects are normally sterilized by chemical preparations. However, chemical preparations have a certain amount of toxicity, which may be left at receptacles or apparatus and harmful to people, meanwhile, the washed off chemical preparations come into the drainpipe, causes pollution to the environment, so an additional filter is needed to provide on the drainpipe.

Therefore, the invention provides a sterilizing instrument to mitigate or obviate the aforementioned problems.

### SUMMARY OF THE INVENTION

The main objective of the present invention is to provide a sterilizing instrument that can provide an excellent sterilizing effect. The sterilizing instrument comprises an ozone producer, a pump, an isopressing tub, and a depressurizer. A water inlet tube is connected to the pump. An air inlet tube is connected between the ozone producer and the water inlet tube. A first transmitting pipe is connected between the pump and the isopressing tub. The isopressing tub has a pressure regulator. A second transmitting pipe is connected between the isopressing tub and the depressurizer. The depressurizer has a valve. The depressurizer is connected with a water outlet tube, wherein a first switch is applied to the water outlet tube to control the water outlet tube and the pump.

Accordingly, a large amount of ozone is dissolved in the water, and the ozone solution is further processed by the depressurizer to generate bubbles in nanometer scale, which contains oxyhydrogen free radicals that can destroy bacteria and virus and is free to pollution to the environment.

Other objectives, advantages and novel features of the invention will become more apparent from the following detailed description when taken in conjunction with the accompanying drawings.

### BRIEF DESCRIPTION OF THE DRAWINGS

FIG. 1 is a side plan view in partial section of a sterilizing instrument in accordance with this invention.

### DETAILED DESCRIPTION OF THE PREFERRED EMBODIMENT

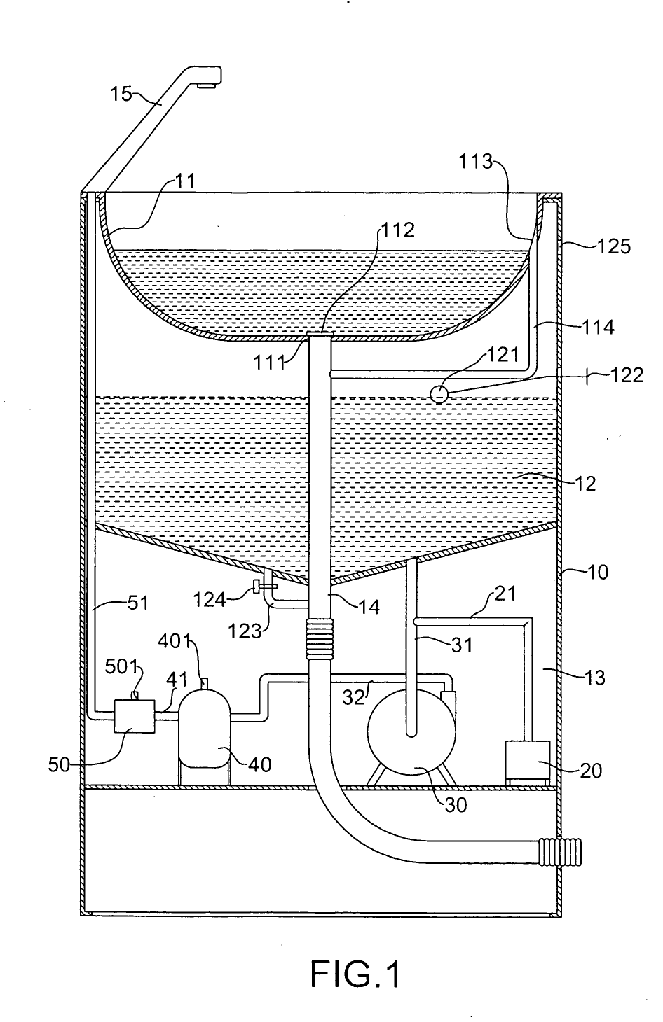

With reference to FIG. 1, a sterilizing instrument in accordance with the present invention has a housing (10), an ozone producer (20), a pump (30), an isopressing tub (40), and a depressurizer (50).

One end of the housing (10) has a washing tub (11) defined therein, an inside of the housing (10) is divided into a first chamber (12) and a second chamber (13). The washing tub (11), the first chamber (12) and the second chamber (13) are not communicated with one each other. A sink drain (14) is mounted in the housing (10), with one end connected to a bottom of the washing tub (11), and the other end extended out of the housing (10) to discharge used water. A faucet (15) is mounted above the washing tub (11), and a first switch is mounted on the faucet to open or close the faucet (15). The faucet (15) is connected to the pump (30) to switch on or off the pump (30). The first switch can be a photoresponse switch, which can open the faucet (15) without contacting the faucet (15).

An outlet (111) is defined at the joint between the sink drain (14) and the washing tub (11). A rubber plug (112) is plugged into the outlet (111) to allow water stored in the washing tub (11). An overflow vent (113) is defined at the wall of the washing tub (11) near top of the washing tub (11). An overflow pipe (114) is connected between the overflow vent (113) and the sink drain (14). If the water level is higher than height of the overflow vent (113), the water will be discharged into the sink drain (14) through the overflow vent (113).

A water level sensor (121) is mounted in the first chamber (12) and is connected with a second switch (122). When the water level in the first chamber (12) is lower than a predetermined level, the second switch (122) is kept at an on position to keep water flowing into the first chamber (12) from the second switch (122). If the water level is higher than the predetermined level, the second switch (122) is switched off to stop water flowing into the first chamber (12). A connecting pipe (123) is connected between the first chamber (12) and the sink drain (14) and is provided with a third switch (124). When the third switch (124) is on, the water in the first chamber (12) can be discharge from the connecting pipe (123) to allow the first chamber (12) to be cleaned. A window (125) is defined at the wall of the first chamber (12) to balance the pressures inside and outside of the first chamber (12).

The ozone producer (20), the pump (30), the isopressing tub (40), and the depressurizer (50) are all mounted in the second chamber (13). The isopressing tub (40) has a pressure regulator (401), and the depressurizer (50) has a valve (501). A water inlet tube (31) is connected between the first chamber (12) and the pump (30), and an air inlet tube (21) is connected between the ozone producer (20) and the water inlet tube (31). A first transmitting pipe (32) is connected between the pump (30) and the isopressing tub (40), and a second transmitting pipe (41) is connected between the isopressing tub (40) and the depressurizer (50). A water outlet tube (51) is connected between the depressurizer (50) and the faucet (15). If more than one faucet (15) is used, more than one water outlet tube (51) is connected to the depressurizer (50).

The ozone generated in the ozone producer (20) is led into the air inlet tube (21), and the water in the first chamber (12) is led into the water inlet tube (31). When the pump (30) is switched on, the water in the water inlet tube (31) and the ozone in the air inlet tube (21) are flowed to the pump (30). The pump (30) boosts the pressure to make the ozone dissolved in the water and to generate air bubbles with micronmeter scale. The dissolved ozone and water produce oxyhydrogen free radicals (OH and HO₂), and the ozone aqueous solution is transmitted to the isopressing tub (40) through the first transmitting pipe (32). The pressure regulator (401) will adjust the pressure of the ozone aqueous solution and discharge redundant air, then the ozone aqueous solution is further transmitted to the depressurizer (50) through the second transmitting pipe (41). During a pressure releasing process in the depressurizer (50), the ozone is encircled by water and millions of air bubbles between 0.1-0.2 micronmeter scale are formed, the air bubbles are jumping and blowing out, and then are divided into smaller bubbles each between 10-20 nanometer. When water with bubbles in nanometer scale is used to wash food, container or skin, the bubbles are so small so to clean objects thoroughly, i.e. the oxyhydrogen free radicals will destroy RNA (ribonucleic acid) and DNA (deoxyribonucleic acid) of bacteria and virus immediately. Moreover, after the sterilizing process, the oxyhydrogen free radicals finally become water and oxygen, which will not pollute environment at all.

It is to be understood, however, that even though numerous characteristics and advantages of the present invention have been set forth in the foregoing description, together with details of the structure and function of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A sterilizing instrument comprising
an ozone producer,
a pump connected to the ozone producer,
an isopressing tub connected to the pump with a first transmitting pipe and having a pressure regulator,
a depressurizer connected to the isopressing tub with a second transmitting pipe and having a valve;
a water inlet tube connected to the pump;
an air inlet tube connected between the ozone producer and the water inlet tube a water outlet tube connected to the depressurizer, wherein a first switch is applied to the water outlet tube to control the water outlet tube and the pump.

2. The sterilizing instrument as claimed in claim 1, wherein the first switch is a photoresponse switch.

3. The sterilizing instrument as claimed in claim 1 further comprising a housing, wherein the ozone producer, the pump, the isopressing tub, and the depressurizer are all mounted in the housing, and the water outlet tube extends out of the housing.

4. The sterilizing instrument as claimed in claim 3, wherein one end of the housing has a washing tub, a sink drain is mounted in the housing, with one end connected to an outlet defined at a bottom of the washing tub, and the other end extending out of the housing; a faucet is mounted above the washing tab, is connected with the water outlet pipe, and the first switch is mounted on the faucet.

5. The sterilizing instrument as claimed in claim 4, wherein the housing has a first chamber and a second chamber, the ozone producer, the pump, the isopressing tub, and the depressurizer are mounted in the second chamber, the first chamber has a second switch to control water to flow into the first chamber, and the water inlet tube is connected with a bottom of the first chamber.

6. The sterilizing instrument as claimed in claim 5, wherein a rubber plug is plugged into the outlet, and an overflow vent is defined at a wall of the washing tub near a top of the washing tub, and an overflow pipe is connected between the overflow vent and the sink drain.

7. The sterilizing instrument as claimed in claim 6, wherein a water level sensor is mounted in the first chamber and is connected with the second switch.

8. The sterilizing instrument as claimed in claim 7, wherein a connecting pipe is connected with the first chamber and the sink drain and is controlled by a third switch.

9. The sterilizing instrument as claimed in claim 8, wherein a window is defined at a wall of the first chamber.
